(19) United States
(12) Patent Application Publication
(10) Pub. No.: US 2010/0144023 A1
(43) Pub. Date: Jun. 10, 2010

(54) Photobioreactor

(75) Inventors: William Patrick WEAVER, Pen Argyl, PA (US); Earl Edwin WISMER, Bethlehem, PA (US)

Correspondence Address: HARNESS, DICKEY & PIERCE, P.L.C., P.O. BOX 828, BLOOMFIELD HILLS, MI, 48303, US

(73) Assignee: Harvel Plastics, Inc., Easton, PA (US)

(21) Appl. No.: 12/371,042

(22) Filed: Feb. 13, 2009

(51) Int. Cl. C12M 3/00; B32B 1/08

(52) U.S. Cl. 435/292.1; 428/36.6

(57) ABSTRACT

A photobioreactor system including a light source, a plurality of interconnected pipes, and a liquid slurry containing an algae disposed within the pipes. The pipes are formed from a translucent polyvinyl chloride material that allows light having a plurality of wavelengths emitted from the light source that stimulate growth of the algae to pass through the material and is resistant to light having a wavelengths emitted from the light source that degrade the material.

### FIELD

The present disclosure relates to the area of photobioreactors.

### BACKGROUND

This section provides background information related to the present disclosure which is not necessarily prior art.

Biochemical engineering is a branch of chemical engineering or biological engineering that mainly deals with the design and construction of unit processes that involve biological organisms or molecules. Biochemical engineering is often used in applications such as food, feed, pharmaceutical, biotechnology, and water treatment industries.

A bioreactor may refer to any device or system that supports a biologically active environment. In one case, a bioreactor is a vessel in which an aerobic or anaerobic chemical process is carried out involving organisms or biochemically active substances derived from such organisms.

Bioreactor design is a relatively complex engineering task. Under optimum conditions, the microorganisms or cells are able to perform their desired function with a 100% rate of success. The bioreactor's environmental conditions like gas flow rates, temperature, pH, dissolved oxygen levels, agitation, and speed/circulation rate, however, must be closely monitored and controlled.

A photobioreactor is a bioreactor that incorporates some type of light source. Photobioreactors are used to grow phototroph small organisms like cyanobacteria, algae, or moss.

In the case of algae, the use of photobioreactors has increased in interest due to the ever-increasing cost of energy. More specifically, various types of algae have been developed that are oil-producing. Because of the limited supply of conventional oil throughout the world, alternative energy sources such as oil-producing algae are increasingly being researched and developed in an attempt avert a global energy shortage. It is desirable, therefore, to develop photobioreactors that are well adapted to produce mass quantities of oil-producing algae to meet the emerging energy needs of today's world.

Various methods to produce oil-producing algae include cultivating the algae in open ponds. Although ponds are not expensive to produce, it is difficult to control temperature fluctuations and water loss that accompanies these ponds. Attempts to control temperature and decrease water loss increase the cost associated with managing these ponds. In addition, unwanted algae growth (i.e., growth of algae that is not oil-producing) is also a concern and lowers the yield of the oil-producing algae.

Accordingly, there is a need for a photobioreactor that is inexpensive to produce, robust and able to withstand the rigors of potentially being erected in a harsh environment such as a desert.

### SUMMARY

This section provides a general summary of the disclosure, and is not a comprehensive disclosure of its full scope or all of its features.

The present disclosure provides a photobioreactor including a light source, a pipe having a wall configured to allow light from the light source to pass therethrough, and a slurry containing an algae. The pipe is formed from a polyvinyl chloride material that resists degradation from predetermined wavelengths of the light.

The material is resistant to UV wavelengths, but the material allows light having a wavelength longer than UV to pass through the wall.

The wall preferably has a thickness and an outer diameter, and a ratio of the outer diameter to the thickness is at least 25 to 1. The ratio increases an amount of light beneficial for the cultivation of the algae allowed to pass through the wall.

The algae may be an energy-producing algae. In particular, the energy-producing algae may be an oil-producing algae.

The pipe may be extruded from a mixture of polyvinyl chloride and a UV-retardant. The polyvinyl chloride material is translucent.

The present disclosure also provides a photobioreactor system including a light source, a plurality of interconnected pipes, and a liquid slurry containing an algae disposed within the pipes. The pipes are formed from a translucent polyvinyl chloride material that allows light having a plurality of first wavelengths emitted from the light source that stimulate growth of the algae to pass through the material and is resistant to light having a plurality of second wavelengths emitted from the light source that degrades the material.

The plurality of second wavelengths include UV light, and the plurality of first wavelengths include visible light.

Each of the pipes preferably includes a wall having a thickness and an outer diameter, and a ratio of the outer diameter to the thickness is at least 25 to 1.

The first wavelengths are shorter than the second wavelengths.

The algae may be an energy-producing algae. In particular, the energy-producing algae may be an oil-producing algae.

The present disclosure also provides a photobioreactor system including a light source, a plurality of interconnected pipes supported by a frame, a liquid slurry containing an oil-producing algae disposed within said pipes, and at least one pump for circulating the slurry through the interconnected pipes. Each of the pipes are formed from a translucent polyvinyl chloride (PVC) material that absorbs ultraviolet wavelengths of light to prevent the PVC material from degrading. The PVC material allows wavelengths of the light greater than UV to pass through the material to cultivate growth of the algae during circulation of the slurry in the pipes.

Each of the pipes includes a wall having a thickness and an outer diameter, and the ratio of the outer diameter to the thickness is preferably at least 25 to 1.

The ratio increases an amount of light beneficial for the cultivation of the algae allowed to pass through the wall.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a perspective view of a photobioreactor according to the present disclosure;

FIG. 2 is a cross-sectional view of a pipe of a photobioreactor according to the present disclosure;

FIGS. 3 and 4 illustrate percent absorbance of various wavelengths of light for polyvinyl chloride (PVC) and UV-inhibited PVC compounds; respectively.

Corresponding reference numerals indicate corresponding parts throughout the several views of the drawings.

### DETAILED DESCRIPTION

Example embodiments will now be described more fully with reference to the accompanying drawings.

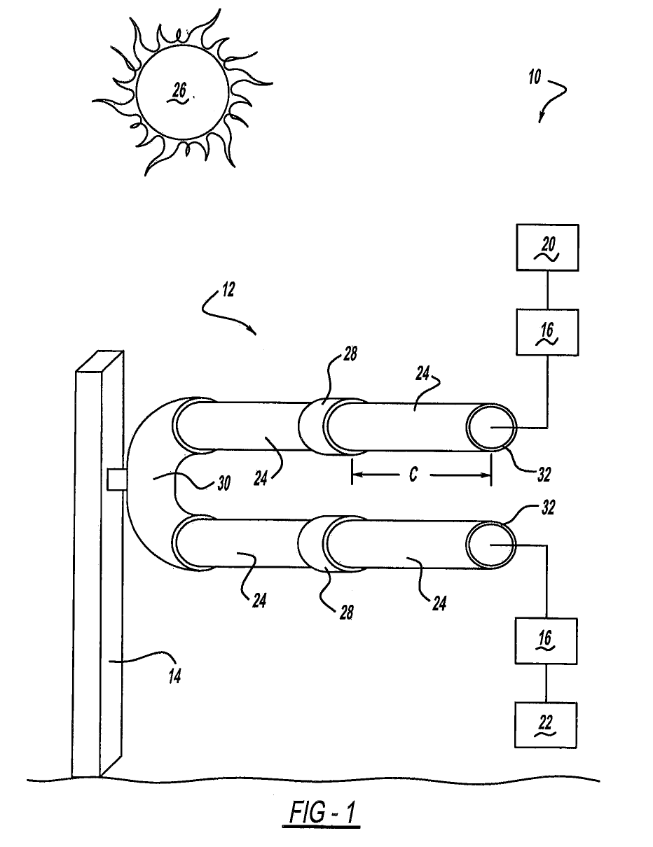

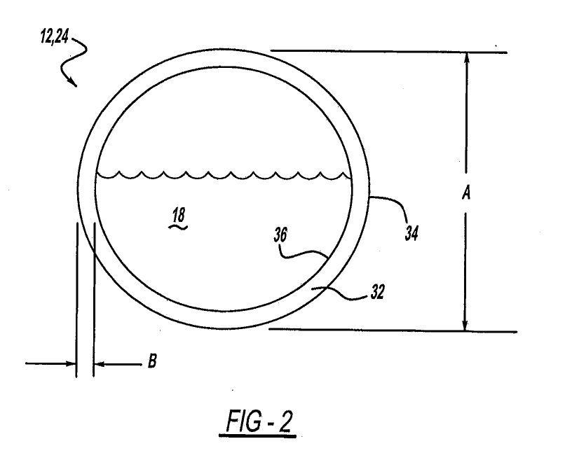

FIGS. 1 and 2 illustrate a photobioreactor system 10 including a photobioreactor 12. In addition to photobioreactor 12, system 10 may include a frame 14 for supporting photobioreactor 12, pumps 16 for circulating a slurry 18 containing an energy-producing organism and water through photobioreactor 12, and storage reservoirs 20 and 22 for feeding the slurry 18 into the photobioreactor 12 and collecting slurry 18 after slurry 18 has passed through photobioreactor 12, respectively. System 10 may also include other units (not shown) such as columns or tanks for oxygen stripping and saturation of slurry 18 with carbon dioxide.

Photobioreactor 12 is formed from plurality of interconnected pipes 24 proximate a light source 26. Pipes 24 are interconnected via couplings 28 and elbows 30. System 10 illustrated in FIG. 1 is preferably used outdoors so that the sun serves as light source 26. It is not out of the scope of the present disclosure, however, to have system 10 used indoors proximate an artificial light source 26 such as a lamp. Although pipes 24 are illustrated to be horizontal relative to the ground, pipes 24 may be slightly askew relative to the horizontal to assist in circulation of slurry 18 therein. Pipes 24 have a closed diameter, i.e. there are no perforations in the walls.

Each of the plurality of pipes 24 is formed from a polyvinyl chloride (PVC) material including an ultraviolet (UV) light inhibitor. An exemplary PVC having a UV-inhibitor is Geon™ Vinyl Rigid Extrusion 87727-0284 sold by PolyOne™. Alternatively, pipes 24 may be formed form a chlorinated polyvinyl chloride (CPVC) material including a UV inhibitior, or mixtures of PVC and CPVC having a UV inhibitor. Pipes 24 formed from either PVC or CPVC having a UV-inhibitor may be used proximate light source 26 that emits light containing UV wavelengths without degrading. In particular, use of pipes 24 formed of PVC or CPVC having a UV-inhibitor enable photobioreactor 12 to be exposed outdoors to sunlight without degrading. It should be understood that although the below disclosure describes the use of PVC having a UV-inhibitor, the below disclosure is equally applicable to the use of CPVC, or mixtures of PVC and CPVC, having a UV-inhibitor and, therefore, the below disclosure should not be limited to solely PVC materials.

UV inhibition is important when pipes 24 are formed from PVC because prolonged exposure of non-UV inhibited transparent PVC to direct sunlight may cause a thin film of degradation on the exposed surface of the pipe over time. This thin film or layer will gradually become visible as discoloration (so-called bleaching) occurs, which prevents passing of sunlight there through. Transparent PVC materials, therefore, are generally not desirable for use in photobioreactor 12 because energy-producing organisms in slurry 18 require light to grow. If light is unable to pass through the thin film of degradation, the processes of photobioreactor 12 will cease unless the thin film of degradation is removed.

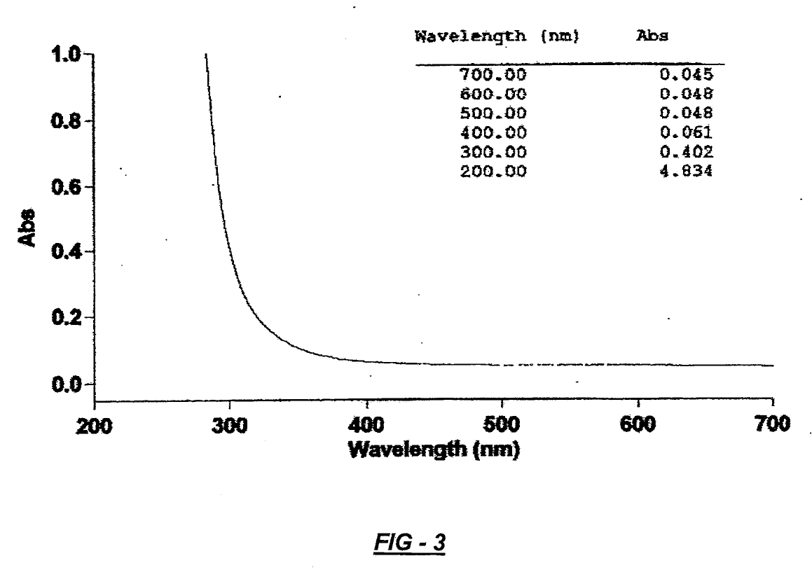

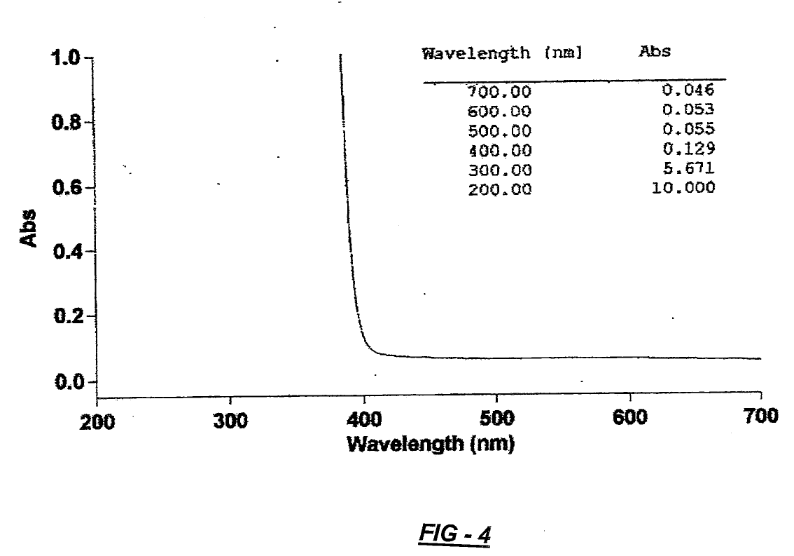

Although pipes 24 formed of PVC having a UV-inhibitor inhibit passing of UV light, light having wavelengths greater than UV (i.e., visible light) is allowed to pass through the material that is beneficial to the cultivation of the energy-producing organisms. FIGS. 3 and 4 illustrate the absorption of light of various wavelengths. FIG. 3 illustrates PVC without a UV-inhibitor, while FIG. 4 illustrates PVC including a UV-inhibitor. As shown by FIG. 3, light having wavelengths of about 300 nm (i.e., UV) and greater is not readily absorbed by the PVC. UV wavelengths, therefore, will degrade the PVC and eventually prevent beneficial wavelengths from passing through walls 32 of pipe 24 and cultivating energy-producing organism growth.

In contrast, now referring to FIG. 4, PVC having a UV-inhibitor is effective in absorbing light having wavelengths up to about 400 nm. Pipe 24 formed of such a material, therefore, will resist degradation and experience increased longevity, which makes PVC having a UV-inhibitor a good material for forming pipes 24 of photobioreactor 12.

Although PVC having a UV-inhibitor is a good material for forming pipes 24 of photobioreactor 12, PVC having a UV-inhibitor may be translucent such that visible light to is allowed to pass through the material, but is diffused so that objects on a side of the material opposite to where light enters are not clearly visible. In other words, although beneficial non-UV wavelengths may pass through walls 32 of pipe 24 and reach slurry 18, the entire amount of beneficial light for cultivating slurry 18 may not pass through wall 32. A thickness of a wall 32 of pipe 24, therefore, should be controlled to maximize the amount of visible light allowed to pass there through to maximize cultivation of the energy-producing organisms without sacrificing durability and longevity of pipe 24.

To maximize the amount of visible light allowed to pass through wall 32 of pipe 24 without sacrificing durability and longevity of pipe 24, a thickness of wall 32 is controlled in relation to an overall diameter of pipe 24. Referring to FIG. 2, a circular pipe 24 is shown in cross-section. Pipe 24 has an overall outer diameter A, and wall 32 has a thickness B measured from outer surface 34 to inner surface 36 of pipe 24. A ratio of outer diameter A to thickness B of wall 32 is preferably at least 25:1. Table 1 (below) shows the outer diameters A of pipes 24 having various size (inches) and a corresponding wall thickness B.

TABLE 1

| | Outside Diameter (A) | | | | |
|---|---|---|---|---|---|
| | | Tolerances | | Wall | |
| | | For | Max. Out of | Thickness (B) | |
| Pipe Size (in) | Average | Avg. | Roundness | Min. | Tolerance |
| 2″ | 2.375 | +/−0.006 | 0.6 | 0.091 | +.020 |
| 3″ | 3.5 | +/−0.008 | 0.6 | 0.135 | +.020 |
| 4″ | 4.5 | +/−0.009 | 0.1 | 0.173 | +.020 |
| 6″ | 6.625 | +/−0.020 | 0.05 | 0.172 | +0.030 |
| 8″ | 8.625 | +/−0.020 | 0.075 | 0.172 | +0.030 |
| 10″ | 10.75 | +/−0.025 | 0.075 | 0.172 | +0.030 |
| 12″ | 12.75 | +/−0.025 | 0.075 | 0.172 | +0.030 |

A ratio of 25:1 for outer diameter A to wall thickness B ensures that the maximum amount of visible light is allowed to pass through wall 32 of pipe 24, while ensuring that pipe 24 remains rigid and able to provide satisfactory mechanical properties such as, but not limited to, impact resistance, tensile modulus, hardness, and thermal deflection. Although pipe 24 is shown to be circular in cross-section, pipes 24 may have any cross-sectional shape without departing from the spirit and scope of the present disclosure. For example, pipes 24 may be square, rectangular, triangular, or any other polygon-shape in cross-section.

As shown in Table 1, UV-resistant PVC pipe 24 may be produced in various sizes. Photobioreactors 12 having various volumes, therefore, may be produced depending on the size or scale of the project desired. A length of pipes 24 is not limited. To assist manufacturing and shipping of pipes 24, however, a length C of pipes 24 may be between 10 and 20 feet.

The use of PVC is also beneficial in that PVC is generally non-reactive, resistant to various pH levels, and also temperature resistant. For example, as stated above, cultivation of the energy-producing organisms may include columns for oxygen stripping and introducing carbon dioxide into the system 10. These processes may cause the pH levels of the system 10 to increase and/or decrease, which requires resistance to varying pH levels. Further, cultivation of the energy-producing organisms may result in various chemical by-products being produced, which may chemically attack pipes 24. PVC being a generally non-reactive material, however, reduces the threat of chemical degradation of pipes 24 during the useful life of system 10. Lastly, as also stated above, system 10 may be erected in a harsh environment such as a desert to make use of abundant sunlight. Because PVC is resistant to higher temperatures, pipes 24 formed from PVC are resistant to failing from prolonged exposure to elevated temperatures.

Pipes 24 may be produced using an extrusion process where UV-resistant PVC is heated and pressurized in a molten state and forced over die and mandrel into the desired pipe geometry. The extruded UV-resistant PVC may then be cooled downstream where final dimensions are realized. Alternatively, PVC and a UV-retardant may be intermixed and subsequently extruded together. Additional methods for producing pipes 24 include injection molding and compression molding. Regardless, any method satisfactory for producing pipes 24 having the appropriate relationship between outer diameter A and wall thickness B is contemplated.

By way of a non-limiting example, the PVC material used for extruding pipe 24 is preferably a mixture of base transparent PVC and/or CPVC resin, impact modifiers, processing aids (i.e. thermal stabilizers) and UV inhibitors that have a minimal impact on the transparency and are compatible with the chemistry of the base PVC/CPVC compound. Alternative materials include a pre-blended rigid Polyvinyl Chloride compound such as PolyOne™ Geon™ Vinyl Rigid Extrusion 87727. The UV inhibitor preferably is a benzophenone compound. Alternative UV inhibitors also include, but are not necessarily limited, to benzotriazole compounds.

In the extrusion process of the transparent UV-resistant PVC pipe 24, raw thermoplastic material in the form of small beads or powder is gravity fed from a top mounted hopper into the barrel of an extruder that contains a rotating screw. The material may be fed as a pre-blended compound into the hopper, or additives such as UV inhibitors may be added separately to the material in the hopper. The material enters the barrel of the extruder through an opening near the rear of the barrel (feed throat) where it comes into contact with the rotating screw. The rotating screw, in combination with a geometry of the screw, forces the beads forward along the screw into a heated barrel under pressure.

The screw geometry, speed of rotation, and heating of the screw are interrelated, specific to the material, and form critical aspects of optimizing the material for processing at this time. The barrel contains heating zones where melt temperatures of the plastic are controlled. Typically, five or more heating zones gradually heat the beads at temperatures ranging from 275° F. to 360° F. along the screw until the material reaches its melt temperature. As the now molten plastic exits the barrel, it travels through a screen pack and breaker plate assembly. The screen pack removes contaminants that may be present in the melt stream, while the breaker plate creates back pressure that is necessary for uniform melting and proper mixing of the molten PVC material. Substantial back pressures ranging from 1,000 to 5,000 psi are necessary during this part of the processing to optimize the physical properties of the material.

After exiting the breaker plate the molten material is forced through a restrictor device and into a die and mandrel (tooling). Temperatures are strictly controlled through multiple die zone heating elements that control temperatures of the melt at different points along the die. The die/tooling design is a critical function of processing this material as it must be designed properly to ensure that the molten plastic flows uniformly over the die to form the shape of the final product. Uniform material flow is critical at this phase to prevent the formation of inherent stresses that can cause warping and deformation when the product is cooled.

As the material forms over the die, the material is forced through a sizing sleeve that determines the final shape and dimensions of the finished pipe 24. At this stage in the extrusion process the product is cooled slowly to set the dimensions of the final shape via a combination of downstream equipment. This is achieved by pulling the extrudate downstream from the die via an electric driven “puller” that pulls the product through various stages of downstream processing including cooling. Cooling is typically achieved by pulling the product through a temperature controlled water bath under a sealed vacuum. The vacuum applied prevents the product from collapsing and maintains the shape of the warm plastic until the dimensions are set by continued cooling (water bath). Various stages of water cooling involving different water bath tanks are utilized in combination to achieve the desired effects (i.e. combinations of water flooding and water spray depending on product size).

Dimensions of the product are continually monitored during this portion of processing to ensure the desired shape and tolerances are maintained. As the material is cooled, the material is pulled through in-line marking equipment where the exterior of the product may be marked for identification either by laser marking or ink jet marking. Markings typically include product name, size, date of manufacture and other critical data necessary for identification and traceability.

After exiting the marking equipment, an in-line counter measures the length of the product being extruded until a pre-determined length is reached. When this occurs a signal is sent from the counter to an in-line circular saw that cuts the product to length as it exits the downstream equipment. The cut-ends of the product are trimmed and blown-out with compressed air by the extruder operator at this time. It is then inspected and measured for conformance to pre-established dimensional requirements by the operator. Finished product is then packaged on-line after inspection.

Product may be set aside periodically from the production area (at pre-determined frequencies) and subjected to complete quality control testing by quality control personnel in conformance with quality assurance test procedures established for the product. Since the extrusion process described is a continuous process involving numerous variables, the physical properties of the material itself, the material feed rates, speed of the screw, screw temperature, processing temperatures, melt temperatures, backpressure, machine speed, puller speed, downstream cooling and other factors are all inter-related and will influence the product quality/end result. Operator skill, equipment used, and processing parameters must be well defined and proven to ensure successful manufacture of the product.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

1. A photobioreactor, comprising:
a pipe having a wall configured to allow light to pass therethrough; and
a slurry containing an algae, wherein said pipe is formed from a polyvinyl chloride material that resists degradation from predetermined wavelengths of light.

2. The photobioreactor of claim 1, wherein said material is resistant to UV wavelengths.

3. The photobioreactor of claim 2, wherein said material allows light having a wavelength longer than UV to pass through said wall.

4. The photobioreactor of claim 1, wherein said wall has a thickness and an outer diameter, and a ratio of said outer diameter to said thickness is at least 25 to 1.

5. The photobioreactor of claim 4, wherein said ratio increases an amount of light beneficial for the cultivation of said algae allowed to pass through said wall.

6. The photobioreactor of claim 1, wherein said algae is an energy-producing algae.

7. The photobioreactor of claim 6, wherein said energy-producing algae is an oil-producing algae.

8. The photobioreactor of claim 1, wherein said pipe is extruded from a mixture of polyvinyl chloride and a UV-retardant.

9. The photobioreactor of claim 1, wherein said polyvinyl chloride material is translucent.

10. A photobioreactor system, comprising:
a plurality of interconnected pipes; and
a liquid slurry containing an algae disposed within said pipes,
said pipes being formed from a translucent polyvinyl chloride material that allows light having a plurality of first wavelengths that stimulate growth of said algae to pass through said material and is resistant to light having a plurality of second wavelengths emitted that degrades said material.

11. The system of claim 10, wherein said plurality of second wavelengths include UV light.

12. The system of claim 11, wherein said plurality of first wavelengths include visible light.

13. The system of claim 10, wherein each of said pipes includes a wall having a thickness and an outer diameter, and a ratio of said outer diameter to said thickness is at least 25 to 1.

14. The system of claim 10, wherein said first wavelengths are shorter than said second wavelengths.

15. The system of claim 10, wherein said algae is an energy- producing algae.

16. The system of claim 15, wherein said energy-producing algae is an oil-producing algae.

17. A photobioreactor system, comprising:
a plurality of interconnected pipes supported by a frame;
a liquid slurry containing an oil-producing algae disposed within said pipes; and
at least one pump for circulating said slurry through said interconnected pipes,
said pipes being formed from a translucent polyvinyl chloride (PVC) material that absorbs ultraviolet wavelengths of light to prevent said PVC material from degrading, said PVC material allowing wavelengths of light greater than UV to pass through said material to cultivate growth of said algae during circulation of said slurry in said pipes.

18. The system of claim 17, wherein each of said pipes includes a wall having a thickness and an outer diameter, and a ratio of said outer diameter to said thickness is at least 25 to 1.

19. The system of claim 18, wherein said ratio increases an amount of light beneficial for the cultivation of said algae allowed to pass through said wall.

20. (canceled)
